(19) United States
(12) Patent Application Publication
(10) Pub. No.: US 2021/0077184 A1
(43) Pub. Date: Mar. 18, 2021

(54) CATHETER WITH THIN-FILM ELECTRODES ON EXPANDABLE MEMBRANE

(71) Applicant: Biosense Webster (Israel) Ltd., Yokneam (IL)

(72) Inventors: Shubhayu Basu, Anaheim, CA (US); Dustin R. Tobey, San Dimas, CA (US); Pieter E. Van Niekerk, Monrovia, CA (US); Cesar Fuentes-Ortega, Pasadena, CA (US)

(21) Appl. No.: 16/997,195

(22) Filed: Aug. 19, 2020

Related U.S. Application Data
(60) Provisional application No. 62/900,749, filed on Sep. 16, 2019.

(51) Int. Cl. A61B 18/14

(52) U.S. Cl. CPC A61B 18/1492; A61B 2018/00214

(57) ABSTRACT

An apparatus includes a catheter and an end effector. The end effector includes an expandable body and a plurality of electrodes deposited on the outer surface of the expandable body. The expandable body is configured to transition between a non-expanded state and an expanded state. The expandable body has an inner surface and an outer surface. The expandable body defines a plurality of openings extending from the inner surface to the outer surface. The electrodes are configured to expand with the expandable body from the non-expanded state to the expanded state. The electrodes including one or more electrodes selected from the group consisting of mapping electrodes that are configured to sense electrical potentials in tissue contacting the mapping electrodes and ablation electrodes that are operable to ablate tissue contacting the ablation electrodes.

### PRIORITY

This application claims priority to U.S. Provisional Pat. App. No. 62/900,749, entitled “Catheter with Thin-Film Electrodes on Expandable Membrane,” filed Sep. 16, 2019, the disclosure of which is incorporated by reference herein in its entirety.

### BACKGROUND

Cardiac arrhythmias, such as atrial fibrillation, occur when regions of cardiac tissue abnormally conduct electric signals. Procedures for treating arrhythmia include surgically disrupting the conducting pathway for such signals. By selectively ablating cardiac tissue by application of energy (e.g., alternating-current or direct-current energy), it may be possible to cease or modify the propagation of unwanted electrical signals from one portion of the heart to another. The ablation process may provide a barrier to unwanted electrical pathways by creating electrically insulative lesions or scar tissue that effectively block communication of aberrant electrical signals across the tissue.

In some procedures, a catheter with one or more electrical electrodes may be used to provide ablation within the cardiovascular system. The catheter may be inserted into a major vein or artery (e.g., the femoral artery) and then advanced to position the electrodes within the heart or in a cardiovascular structure adjacent to the heart (e.g., the pulmonary vein). The electrodes may be placed in contact with cardiac tissue or other vascular tissue and then activated with electrical energy to thereby ablate the contacted tissue. In some cases, the electrodes may be bipolar. In some other cases, a monopolar electrode may be used in conjunction with a ground pad or other reference electrode that is in contact with the patient.

Examples of ablation catheters are described in U.S. Pub. No. 2013/0030426, entitled “Integrated Ablation System using Catheter with Multiple Irrigation Lumens,” published Jan. 31, 2013, the disclosure of which is incorporated by reference herein, in its entirety; U.S. Pub. No. 2017/0312022, entitled “Irrigated Balloon Catheter with Flexible Circuit Electrode Assembly,” published Nov. 2, 2017, the disclosure of which is incorporated by reference herein, in its entirety; U.S. Pub. No. 2018/0071017, entitled “Ablation Catheter with a Flexible Printed Circuit Board,” published Mar. 15, 2018, the disclosure of which is incorporated by reference herein, in its entirety; U.S. Pub. No. 2018/0056038, entitled “Catheter with Bipole Electrode Spacer and Related Methods,” published Mar. 1, 2018, the disclosure of which is incorporated by reference herein, in its entirety; U.S. Pat. No. 10,130,422, entitled “Catheter with Soft Distal Tip for Mapping and Ablating Tubular Region,” issued Nov. 20, 2018, the disclosure of which is incorporated by reference herein, in its entirety; U.S. Pat. No. 8,956,353, entitled “Electrode Irrigation Using Micro-Jets,” issued Feb. 17, 2015, the disclosure of which is incorporated by reference herein, in its entirety; and U.S. Pat. No. 9,801,585, entitled “Electrocardiogram Noise Reduction,” issued Oct. 31, 2017, the disclosure of which is incorporated by reference herein, in its entirety.

Some catheter ablation procedures may be performed after using electrophysiology (EP) mapping to identify tissue regions that should be targeted for ablation. Such EP mapping may include the use of sensing electrodes on a catheter (e.g., the same catheter that is used to perform the ablation or a dedicated mapping catheter). Such sensing electrodes may monitor electrical signals emanating from conductive endocardial tissues to pinpoint the location of aberrant conductive tissue sites that are responsible for the arrhythmia. Examples of an EP mapping system are described in U.S. Pat. No. 5,738,096, entitled “Cardiac Electromechanics,” issued Apr. 14, 1998, the disclosure of which is incorporated by reference herein, in its entirety. Examples of EP mapping catheters are described in U.S. Pat. No. 9,907,480, entitled “Catheter Spine Assembly with Closely-Spaced Bipole Microelectrodes,” issued Mar. 6, 2018, the disclosure of which is incorporated by reference herein, in its entirety; U.S. Pat. No. 10,130,422, entitled “Catheter with Soft Distal Tip for Mapping and Ablating Tubular Region,” issued Nov. 20, 2018, the disclosure of which is incorporated by reference herein, in its entirety; and U.S. Pub. No. 2018/0056038, entitled “Catheter with Bipole Electrode Spacer and Related Methods,” published Mar. 1, 2018, the disclosure of which is incorporated by reference herein, in its entirety.

In addition to using EP mapping, some catheter ablation procedures may be performed using an image guided surgery (IGS) system. The IGS system may enable the physician to visually track the location of the catheter within the patient, in relation to images of anatomical structures within the patient, in real time. Some systems may provide a combination of EP mapping and IGS functionalities, including the CARTO 3® system by Biosense Webster, Inc. of Irvine, Calif. Examples of catheters that are configured for use with an IGS system are disclosed in U.S. Pat. No. 9,480,416, entitled “Signal Transmission Using Catheter Braid Wires,” issued Nov. 1, 2016, the disclosure of which is incorporated by reference herein, in its entirety; and various other references that are cited herein.

While several catheter systems and methods have been made and used, it is believed that no one prior to the inventors has made or used the invention described in the appended claims.

### BRIEF DESCRIPTION OF THE DRAWINGS

The drawings and detailed description that follow are intended to be merely illustrative and are not intended to limit the scope of the invention as contemplated by the inventors.

FIG. 1 depicts a schematic view of a medical procedure in which a catheter of a catheter assembly is inserted in a patient;

FIG. 2A depicts a top plan view of the catheter assembly of FIG. 1, with an end effector in a non-expanded state;

FIG. 2B depicts a top plan view of the catheter assembly of FIG. 1, with the end effector in an expanded state;

FIG. 3 depicts an enlarged perspective view of the end effector of FIG. 2A in the expanded state;

FIG. 4 depicts an enlarged perspective view of an example of a variation of the end effector of FIG. 2A, with integral resilient elements assisting in urging the end effector to the expanded state;

FIG. 5 depicts a cross-sectional view of a portion of the end effector of FIG. 2A;

FIG. 6 depicts an enlarged perspective view of an example of an alternative end effector that may be incorporated into the catheter assembly of FIG. 1;

FIG. 7 depicts an enlarged perspective view of another example of an alternative end effector that may be incorporated into the catheter assembly of FIG. 1;

FIG. 8 depicts an enlarged perspective view of another example of an alternative end effector that may be incorporated into the catheter assembly of FIG. 1;

FIG. 9 depicts a top plan view of a flattened body of another example of an alternative end effector that may be incorporated into the catheter assembly of FIG. 1;

FIG. 10 depicts an enlarged perspective view of the body of FIG. 9 incorporated into an end effector for the catheter assembly of FIG. 1; and

FIG. 11 depicts a cross-sectional view of a portion the end effector of FIG. 10.

### DETAILED DESCRIPTION

The following description of certain examples of the invention should not be used to limit the scope of the present invention. The drawings, which are not necessarily to scale, depict selected embodiments and are not intended to limit the scope of the invention. The detailed description illustrates by way of example, not by way of limitation, the principles of the invention. Other examples, features, aspects, embodiments, and advantages of the invention will become apparent to those skilled in the art from the following description, which is by way of illustration, one of the best modes contemplated for carrying out the invention. As will be realized, the invention is capable of other different or equivalent aspects, all without departing from the invention. Accordingly, the drawings and descriptions should be regarded as illustrative in nature and not restrictive.

Any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±10% of the recited value, e.g. “about 90%” may refer to the range of values from 81% to 99%. In addition, as used herein, the terms “patient,” “host,” “user,” and “subject” refer to any human or animal subject and are not intended to limit the systems or methods to human use, although use of the subject invention in a human patient represents a preferred embodiment.

### I. Overview of Example of a Catheter System

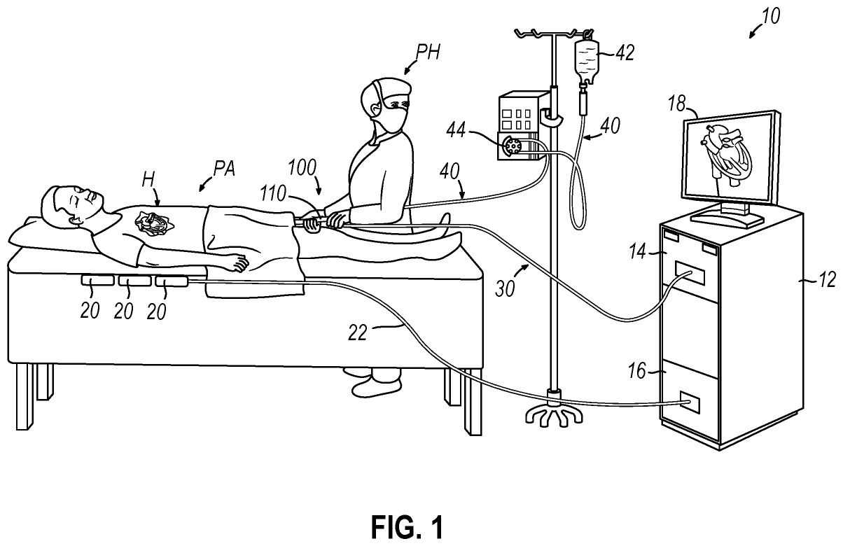

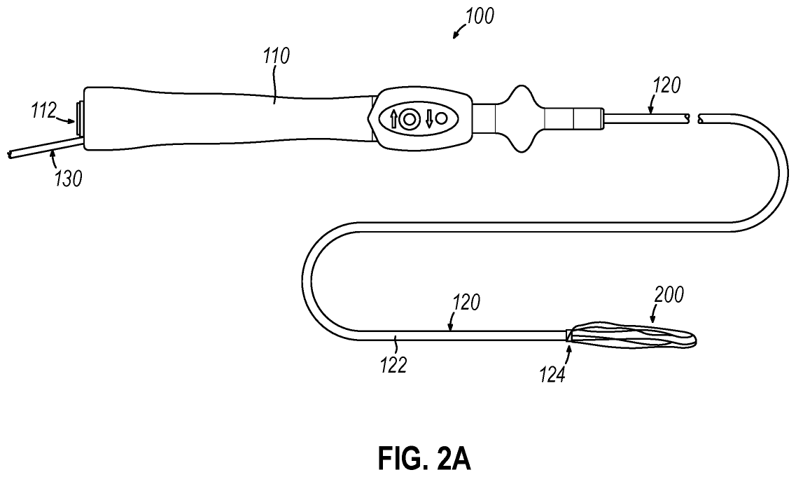

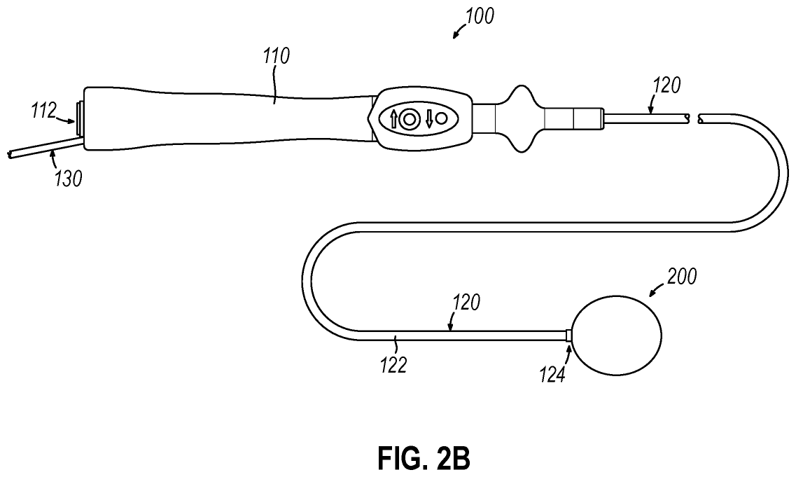

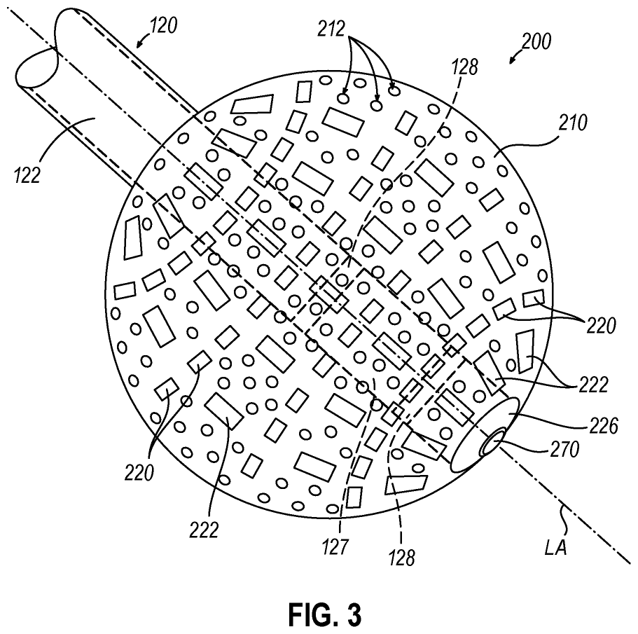

FIG. 1 shows an example of a medical procedure and associated components of a cardiac ablation system. In particular, FIG. 1 shows a physician (PH) grasping a handle (110) of a catheter assembly (100), with an end effector (200) of a flexible catheter (120) (shown in FIGS. 2A-3 but not shown in FIG. 1) of catheter assembly (100) disposed in a patient (PA) to map or ablate tissue in or near the heart (H) of the patient (PA). As shown in FIGS. 2A-3, catheter (120) includes an outer sheath (122), with end effector (200) being disposed at or near a distal end (124) of outer sheath (122). Catheter assembly (100) is coupled with a guidance and drive system (10) via a cable (30). Catheter assembly (100) is also coupled with a fluid source (42) via a fluid conduit (40), though this is merely optional. A set of field generators (20) are positioned underneath the patient (PA) and are also coupled with guidance and drive system (10) via a cable (22).

Guidance and drive system (10) of the present example includes a console (12) and a display (18). Console (12) includes a first driver module (14) and a second driver module (16). First driver module (14) is coupled with catheter assembly (100) via cable (30). In some variations, first driver module (14) is operable to receive EP mapping signals obtained via electrodes (250) of end effector (200) as described in greater detail below. Console (12) includes a processor (not shown) that processes such EP mapping signals and thereby provides EP mapping as is known in the art. In addition, or in the alternative, first driver module (14) may be operable to provide electrical power to electrodes (260) of end effector (200) to thereby ablate tissue. In some versions, first driver module (14) is also operable to receive position indicative signals from one or more position sensors (270) in end effector (200), as will be described in greater detail below. In such versions, the processor of console (12) is also operable to process the position indicative signals from a position sensor (270) to thereby determine the position of the end effector (200) of catheter (120) within the patient (PA).

Second driver module (16) is coupled with field generators (20) via cable (22). Second driver module (16) is operable to activate field generators (20) to generate an alternating magnetic field around the heart (H) of the patient (PA). For instance, field generators (20) may include coils that generate alternating magnetic fields in a predetermined working volume that contains the heart (H).

Display (18) is coupled with the processor of console (12) and is operable to render images of patient anatomy. Such images may be based on a set of preoperatively or intraoperatively obtained images (e.g., a CT or MM scan, 3-D map, etc.). The views of patient anatomy provided through display (18) may also change dynamically based on signals from the position sensor (270) of end effector (200). For instance, as end effector (200) of catheter (120) moves within the patient (PA), the corresponding position data from the position sensor (270) may cause the processor of console (12) to update the patient anatomy views in display (18) in real time to depict the regions of patient anatomy around end effector (200) as end effector (200) moves within the patient (PA). Moreover, the processor of console (12) may drive display (18) to show locations of aberrant conductive tissue sites, as detected via EP mapping with end effector (200). By way of example only, the processor of console (12) may drive display (18) to superimpose the locations of aberrant conductive tissue sites on the images of the patient's anatomy, such as by superimposing an illuminated dot, a crosshair, or some other form of visual indication of aberrant conductive tissue sites.

The processor of console (12) may also drive display (18) to superimpose the current location of end effector (200) on the images of the patient's anatomy, such as by superimposing an illuminated dot, a crosshair, a graphical representation of end effector (200), or some other form of visual indication. Such a superimposed visual indication may also move within the images of the patient anatomy on display (18) in real time as the physician moves end effector (200) within the patient (PA), thereby providing real-time visual feedback to the operator about the position of end effector (200) within the patient (PA) as end effector (200) moves within the patient (PA). The images provided through display (18) may thus effectively provide a video tracking the position of end effector (200) within a patient (PA), without necessarily having any optical instrumentation (i.e., cameras) viewing end effector (200). In the same view, display (18) may simultaneously visually indicate the locations of aberrant conductive tissue sites detected through the EP mapping as described herein. The physician (PH) may thus view display (18) to observe the real time positioning of end effector (200) in relation to the mapped aberrant conductive tissue sites and in relation to images of the adjacent anatomical structures in the patient (PA).

Fluid source (42) of the present example includes a bag containing saline or some other suitable irrigation fluid. Conduit (40) includes a flexible tube that is further coupled with a pump (44), which is operable to selectively drive fluid from fluid source (42) to catheter assembly (100). In some variations, conduit (40), fluid source (42), and pump (44) are omitted entirely. In versions where these components are included, end effector (200) may be configured to communicate irrigation fluid from fluid source (42) to the target site in the patient. Such irrigation may be provided in accordance with the teachings of any of the various patent references cited herein; or in any other suitable fashion as will be apparent to those skilled in the art in view of the teachings herein.

FIGS. 2A-2B show ablation catheter assembly (100) in greater detail. As shown, catheter (120) extends distally from handle (110); while a fluid connector assembly (130) extends proximally from handle (110). Fluid connector assembly (130) is configured to couple with conduit (40) to thereby provide a path for irrigation fluid to be communicated from fluid source (42) to end effector (200). As fluid irrigation is a merely optional feature of ablation catheter assembly (100), fluid connector assembly (130) may be omitted if desired. Handle (110) of the present example also includes a socket (112), which is configured to receive a plug (not shown) on the distal end of cable (30) to thereby provide a path for electrical communication between console (12) and end effector (200). Various suitable components and configurations that may be used to form these components will be apparent to those skilled in the art in view of the teachings herein.

### II. Example of an End Effector with Expandable Assembly Having Flex Circuits

As also shown in FIGS. 2A-2B and 3, end effector (200) is positioned at the distal end (124) of catheter (120). While FIGS. 2A-2B show end effector (200) in schematic form, FIG. 3 shows end effector (200) in greater detail. End effector (200) is configured to transition between a non-expanded configuration (FIG. 2A) and an expanded configuration (FIG. 2B). In some versions, end effector (200) is configured to have a size less than or equal to approximately 6 French when in the non-expanded configuration. End effector (200) may be kept in the non-expanded configuration as catheter (120) is inserted into the patient (PA). Once end effector (200) reaches a target site in the patient, end effector (200) may be transitioned to the expanded configuration. In some versions, end effector (200) is positioned within a sheath (not shown) during transit toward the target site in the patient (PA) while end effector (200) is in the non-expanded configuration. The sheath may be a slidably disposed over catheter (120). Once distal end (124) reaches the target site, end effector (200) may be positioned distally relative to the distal end of the sheath and may then be transitioned to the expanded configuration. Some examples of how end effector (200) may be transitioned between the non-expanded configuration and the expanded configuration will be described in greater detail below, while other examples will be apparent to those skilled in the art in view of the teachings herein.

End effector (200) is positioned distally of distal end (124) of outer sheath (122). In some scenarios, end effector (200) is slidably disposed in outer sheath (122); and end effector (200) and outer sheath (122) are advanced together into a lumen (e.g., artery, vein, etc.) of the patient (PA) until distal end (124) is near a target site in the patient (PA). End effector (200) may be initially retracted proximally relative to distal end (124) as the combination of end effector (200) and outer sheath (122) are advanced into position. Once reaching the target site, end effector (200) may be advanced distally as outer sheath (120) is held stationary, to thereby advance end effector (200) from distal end (124). Alternatively, end effector (200) may be held stationary as outer sheath (122) is retracted proximally to reveal end effector (200).

As shown in FIG. 3, end effector (200) of this example includes an inflatable body (210), a plurality of mapping electrodes (220), a plurality of ablation electrodes (222), a central shaft (126), and a distal hub (270). Inflatable body (210) is in the form of a membrane that defines a plurality of openings (212). Openings (212) are large enough to allow fluid to pass through openings (212) while being small enough to allow inflatable body (210) to achieve and maintain an expanded state when inflatable body is filled with an inflation fluid (e.g., saline, etc.). In some versions, the same fluid that is used to inflate inflatable body (210) is expelled through openings (212) to provide irrigation at a targeted site in the patient (PA). For instance, fluid from fluid source (42) may be expelled through openings (212). In addition, or in the alternative, the blood of the patient (PA) may enter the interior of end effector (200) via openings (212) to reach reference electrodes (128) coaxially mounted to central shaft (126). Such reference electrodes (128) will be described in greater detail below.

As another merely illustrative alternative, inflatable body (210) may include two layers, with a fluid-tight space between the layers that receives an inflation fluid, such that the inflation fluid is not expelled through openings (212). In some such versions, irrigation fluid from fluid source (42) is communicated to the interior of inflatable body (210) via fluid conduit (40); and is expelled out through openings (212). It should also be understood that openings (212) may be omitted in some versions. By way of further example only, inflatable body (210) may be made of a non-extensible material. Alternatively, inflatable body (210) may be made of an extensible material. In some variations, body (210) lacks openings (212). In such versions (and in versions where openings (212) are present), irrigation fluid may be expelled from end effector (200) via central shaft (126). For instance, central shaft (126) may include at least one distal opening or lateral openings that are configured to expel irrigation fluid.

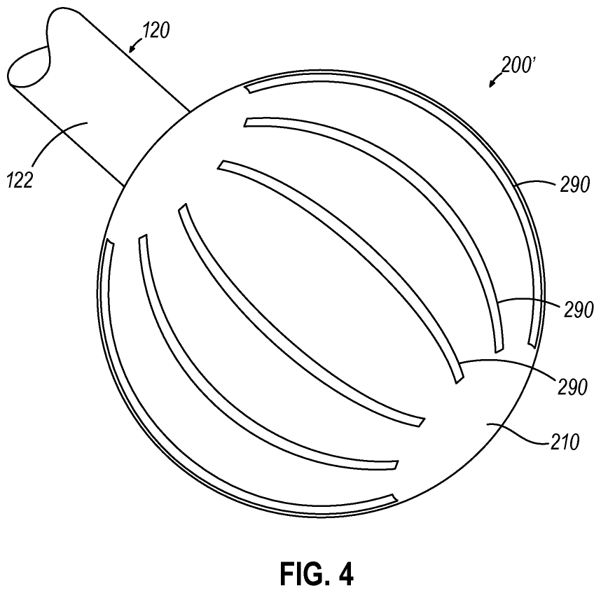

FIG. 4 shows an example of a variation of end effector (200). In this example, end effector (200′) includes a plurality of resilient strips (290) secured to (or otherwise incorporated into) body (210). The other components of end effector (200) are omitted from the depiction of end effector (200′) in FIG. 4 for the sake of simplicity, it being understood that the only difference between end effector (200) and end effector (200′) is the inclusion of resilient strips (290) in end effector (200′). Resilient strips (290) are configured to resiliently bias body (210) toward the expanded configuration shown in FIG. 4. In some such versions, body (210) is not filled with any kind of fluid to drive expansion, such that resilient strips (290) alone provide enough bias for end effector (200) to achieve the expanded state. In some other versions, resilient strips (290) cooperate with the inflating fluid to thereby supplement the expansion of body (210) as induced by the inflating fluid.

By way of example only, resilient strips (290) may comprise nitinol. By way of further example only, resilient strips (290) may be deposited directly onto the inner surface or the outer surface of body (210). For instance, resilient strips (290) may be formed of nitinol that is vapor deposited on inflatable body (210) as a thin film (e.g., through a physical vapor deposition (PVD) process). By way of example only, such a PVD process may be carried out in accordance with at least some of the teachings of International Patent Pub. No. WO 2015/117908, entitled “Medical Device for Ablating Tissue Cells and System Comprising a Device of This Type,” published Aug. 13, 2015, the disclosure of which is incorporated by reference herein, in its entirety; at least some of the teachings of German Patent Pub. No. 102017130152, entitled “Method for Operating a Multi-Layer Structure,” published Jan. 3, 2019, the disclosure of which is incorporated by reference herein, in its entirety; or at least some of the teachings of U.S. Pat. No. 10,061,198, entitled “Method for Producing a Medical Device or a Device with Structure Elements, Method for Modifying the Surface of a Medical Device or of a Device with Structure Elements, Medical Device and Laminated Composite with a Substrate,” published Aug. 28, 2018, the disclosure of which is incorporated by reference herein, in its entirety. Other methods may also be employed to deposit resilient strips (290), including but not limited to sputter deposition, chemical vapor deposition (CVD), thermal deposition, etc. It should also be understood that resilient strips (290) may be formed of other materials, in addition to or in lieu of being formed of nitinol.

In the example shown in FIG. 3, mapping electrodes (220) are arranged in generally circumferential arrays that extend along respective latitudinal paths, with such latitudinal paths being longitudinally spaced apart from each other. Also in the example shown in FIG. 3, ablation electrodes (222) are arranged in generally longitudinal arrays that extend along respective longitudinal paths, with such longitudinal paths being angularly spaced apart from each other. Of course, these arrangements of electrodes (220, 222) are merely illustrative examples. Electrodes (220, 222) may be located in any other suitable positions and arrangements as will be apparent to those skilled in the art in view of the teachings herein.

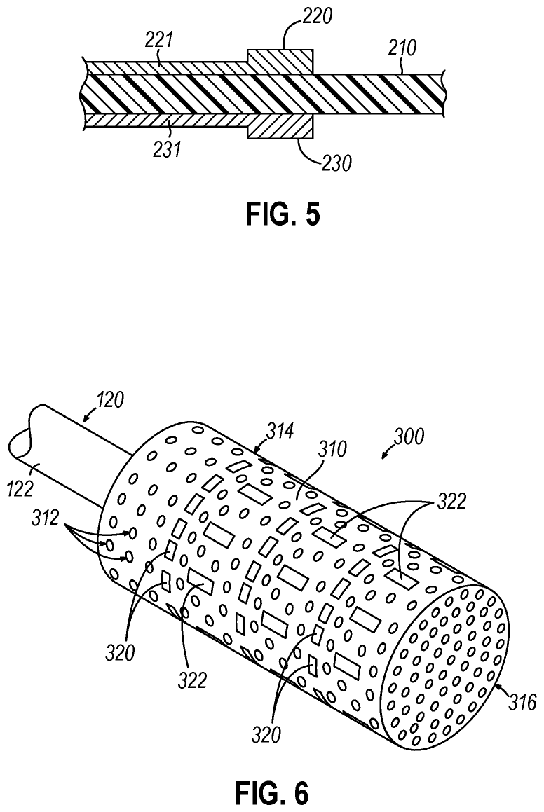

Electrodes (220, 222) may each be printed directly on body (210) or otherwise be directly applied to body (210). FIG. 5 shows an example where electrode (220) and a corresponding conductive trace (221) are applied directly onto body (210). By way of example only, electrode (220) and trace (221) may be applied to body (210) using a physical vapor deposition (PVD) process, sputter deposition, chemical vapor deposition (CVD), thermal deposition, or any other suitable process. For instance, any of the processes described above in relation to the application of resilient strips (290) to body (210) may also be utilized to apply electrodes (220, 222) to body (210). Each electrode (220, 222) may be similarly applied to body (210) with corresponding traces. In some versions, a layer of electrically insulating material is further applied on or between the traces (221) of electrodes (220, 222).

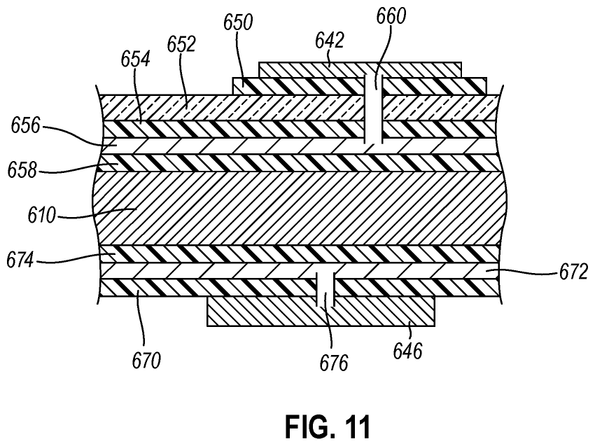

In versions of end effector (200′) where resilient strips (290) are included, electrodes (220, 222) may be applied separately from resilient strips (290). In other words, electrodes (220, 222) may be spaced apart from resilient strips (290) on the surface of body (210). In some other versions of end effector (200′) where resilient strips (290) are included, electrodes (220, 222) may be applied directly onto resilient strips (290). In some such versions, electrodes (220, 222) may be applied to resilient strips (290) in a manner similar to that described below in the context of electrodes (642, 644) being applied to strip body (610) as shown in FIG. 11.

Mapping electrodes (220) are configured to provide EP mapping by contacting tissue and picking up potentials from the contacted tissue (e.g., to provide an electrocardiogram signal). In some versions, mapping electrodes (220) cooperate in bipolar pairs during such mapping procedures. Thus, pair of mapping electrodes (220) may be considered as collectively forming a single “sensor.” Each mapping electrode (220) may be coupled with a corresponding trace (221) (FIG. 5) or other electrical conduit, thereby enabling signals picked up by mapping electrodes (220) to be communicated back through electrical conduits (not shown) in catheter (120) to console (12), which may process the signals to provide EP mapping to thereby identify locations of aberrant electrical activity within the cardiac anatomy. This may in turn allow the physician (PH) to identify the most appropriate regions of cardiac tissue to ablate (e.g., with electrical energy, cryoablation, etc.), to thereby prevent or at least reduce the communication of aberrant electrical activity across the cardiac tissue.

As noted above, and as shown in FIG. 3, end effector (200) further includes a pair of reference electrodes (128) coaxially mounted to central shaft (126). Such reference electrodes (128) may be utilized in conjunction with electrodes (220) during an EP mapping procedure. For instance, reference electrodes (128) may be utilized to pick up reference potentials from blood or saline that passes through the interior of end effector (200) via openings (212) during an EP mapping procedure. Such reference potentials may be used to reduce noise or far field signals, as is known in the art. In the present example, since reference electrodes (128) are effectively contained within the interior of inflatable body (210), inflatable body (210) will prevent tissue from contacting reference electrodes (128) during use of end effector (200) in an EP mapping procedure; while still allowing blood and saline to flow freely through end effector (200) to reach reference electrodes (128). Alternatively, reference electrodes (128) may be positioned in any other suitable location(s); and any suitable number of reference electrodes (128) may be provided.

FIG. 5 shows another example where a reference electrode (230) is positioned on the interior side of inflatable body (210), opposite to electrode (220). In this example, reference electrode (230) and a corresponding trace (231) is applied directly to inflatable body (210), such as by using a physical vapor deposition (PVD) process, sputter deposition, chemical vapor deposition (CVD), thermal deposition, or any other suitable process. In some versions, a layer of electrically insulating material is further applied on or between the traces (231) of reference electrodes (230). Reference electrode (230) may operate similar to reference electrode (128) described above, such that reference electrodes (230) may be utilized to pick up reference potentials from blood or saline that passes through the interior of end effector (200) via openings (212) during an EP mapping procedure. Since reference electrodes (230) would be positioned within the interior of end effector (200), body (210) will prevent tissue from contacting reference electrodes (230) during use of end effector (200) in an EP mapping procedure; while still allowing blood and saline to flow freely through end effector (200) to reach reference electrodes (230). Trace (231) forms part of the path for signals picked up by reference electrode (230) to reach console (12). In some versions, just one single reference electrode (230) is positioned opposite to each mapping electrode (220). Alternatively, reference electrodes (230) may have any other suitable spatial or structural relationships with mapping electrodes (230)

In the present example, as shown in FIG. 3, ablation electrodes (222) are larger than mapping electrodes (220) in this example. Ablation electrodes (222) may be used to apply electrical energy to tissue that is in contact with electrodes (222), to thereby ablate the tissue. Each ablation electrode (222) may be coupled with a corresponding trace (e.g., similar to the arrangement shown in FIG. 5) or other electrical conduit, thereby enabling console (12) to communicate electrical energy through electrical conduits (not shown) in catheter (120) to the traces or other conduits of to reach ablation electrodes (222). In some scenarios, only one, only two, or some other relatively small number of ablation electrodes (222) would be activated to apply electrical energy to tissue at any given moment. As with mapping electrodes (220), the number and positioning of ablation electrodes (222) as shown in FIG. 3 is merely illustrative. Any other suitable number or positioning may be used for ablation electrodes (222). As yet another merely illustrative variation, ablation electrodes (222) may be omitted from end effector (200). In some such variations, mapping electrodes (220) are still included on end effector (200). As used herein, the term “ablate” is intended to cover either radio-frequency ablation or irreversible electroporation.

By way of example only, electrodes (128, 220, 222, 230) may be formed of nitinol, platinum, gold, or any other suitable biocompatible material. In some versions, electrodes (128, 220, 222, 230) are formed of an extensible material and are thereby configured to expand with body (210). Electrodes (220, 222, 230) may be applied directly to body (210) using a physical vapor deposition (PVD) process, sputter deposition, chemical vapor deposition (CVD), thermal deposition, or any other suitable process. Electrodes (128, 220, 222, 230) may include various coatings, if desired. For instance, electrodes (220) may include a coating that is selected to improve the signal-to-noise ratio of signals from electrodes (220). Such coatings may include, but need not be limited to, iridium oxide (IrOx) coating, poly(3,4-ethylenedioxythiophene) (PEDOT) coating, Electrodeposited Iridium Oxide (EIROF) coating, Platinum Iridium (PtIr) coating, or any other suitable coating. Various suitable kinds of coatings that may be used for electrodes (128, 220, 222, 230) will be apparent to those skilled in the art in view of the teachings herein.

By way of further example only, electrodes (220) may be spaced and arranged in accordance with at least some of the teachings of U.S. Provisional Patent App. No. 62/819,738, entitled “Electrode Configurations for Diagnosis of Arryhtmias,” filed Mar. 18, 2019, the disclosure of which is incorporated by reference herein, in its entirety. For instance, electrodes (220) may be spaced and arranged in accordance with FIGS. 13A, 13B, 13C, and 13D) of U.S. Provisional Patent App. No. 62/819,738. Electrodes (226, 230, 232) may be further constructed and operable in accordance with at least some of the teachings of U.S. Pub. No. 2017/0312022, entitled “Irrigated Balloon Catheter with Flexible Circuit Electrode Assembly,” published Nov. 2, 2017, the disclosure of which is incorporated by reference herein, in its entirety.

End effector (200) of the present example further includes a position sensor (270) located at hub (226) at the distal end of end effector (200). Position sensor (270) is operable to generate signals that are indicative of the position and orientation of end effector (200) within the patient (PA). By way of example only, position sensor (270) may be in the form of a wire coil or a plurality of wire coils (e.g., three orthogonal coils) that are configured to generate electrical signals in response to the presence of an alternating electromagnetic field generated by field generators (20). Position sensor (270) may be coupled with wire, a trace, or any other suitable electrical conduit along or otherwise through catheter (120), thereby enabling signals generated by position sensor (270) to be communicated back through electrical conduits (not shown) in catheter (120) to console (12). Console (12) may process the signals from position sensor (270) to identify the position of end effector (200) within the patient (PA). Other components and techniques that may be used to generate real-time position data associated with end effector (200) may include wireless triangulation, acoustic tracking, optical tracking, inertial tracking, and the like. While position sensor (270) is shown as being located on hub (226) in this example, one or more position sensors (270) may be incorporated elsewhere into body (210) or on body (210), in addition to or in lieu of being incorporated into hub (226). In some versions, position sensor (270) may be omitted entirely from end effector (200).

During use of ablation catheter assembly (100), catheter (120) may be advanced to position end effector (200) near a targeted cardiovascular structure (e.g., a chamber of the heart (H), the pulmonary vein, etc.) while end effector (200) is in the non-expanded configuration. End effector (200) may then be expanded to bring electrodes (220, 222) into contact with the tissue of the targeted cardiovascular structure. In some versions, the operator may selectively inflate end effector (200) to provide a desired degree of expansion, with the degree of expansion being selected based on the dimensions or structural configuration of the particular anatomical structure that is being targeted; or based on whether end effector (200) is being used in a mapping procedure or an ablation procedure. For instance, the physician (PH) may provide a larger amount of expansion of end effector (200) when end effector (200) is in a chamber of the heart (H); and a smaller amount of expansion of end effector (200) when end effector (200) is in the pulmonary vein. As another merely illustrative example, the physician (PH) may provide a larger amount of expansion of end effector (200) when end effector (200) being used to perform EP mapping (e.g., expanding end effector to a diameter from approximately 2.5 cm to approximately 3 cm); and a smaller amount of expansion of end effector (200) when end effector (200) is being used to perform ablation (e.g., expanding end effector to a diameter from approximately 5 mm to approximately 9 mm). Other suitable ways in which end effector (200) may be used will be apparent to those skilled in the art in view of the teachings herein.

### III. Examples of Alternative End Effector Membrane Shapes

While end effector (200) of the examples described above presents a generally bulbous or spherical shape when end effector (200) is in the expanded state, variations of end effector (200) may present other kinds of shapes when in the expanded state. Several examples of alternative shapes will be described in greater detail below.

### A. Example of End Effector with Cylindraceous Shape

FIG. 6 shows an example of an end effector (300) located at distal end (124) of catheter (120) in place of end effector (200). End effector (300) of this example may be configured and operable just like end effector (200) described above, except for the differences described below. Like end effector (200), end effector (300) of this example includes an inflatable body (310) (e.g., in the form of an expandable membrane), a set of mapping electrodes (320), and a set of ablation electrodes (322). While not shown, end effector (300) may further include a central shaft like central shaft (126); and in some versions, may further include reference electrodes like reference electrodes (128, 230). Mapping electrodes (320) are arranged in generally circumferential arrays that extend along respective latitudinal paths, with such latitudinal paths being longitudinally spaced apart from each other; and are otherwise configured and operable just like mapping electrodes (220). Ablation electrodes (322) are arranged in generally longitudinal arrays that extend along respective longitudinal paths, with such longitudinal paths being angularly spaced apart from each other; and are otherwise configured and operable just like ablation electrodes (222).

By way of example only, electrodes (320, 322) may be formed of nitinol, platinum, gold, or any other suitable material. In some versions, electrodes (320, 322) are formed of an extensible material and are thereby configured to expand with body (310). Electrodes (320, 322) may be applied directly to body (310) using a physical vapor deposition (PVD) process, sputter deposition, chemical vapor deposition (CVD), thermal deposition, or any other suitable process.

Like inflatable body (210), inflatable body (310) includes openings (312) and is operable to transition between a non-expanded state and an expanded state. However, unlike inflatable body (210), inflatable body (310) has a cylindraceous shape when in the expanded state. In this example, electrodes (320, 322) are only on the longitudinally extending portion (314) of inflatable body (310), such that electrodes (320, 322) do not extend along a flat distal face (316) of inflatable body (310). In some other versions, electrodes (320, 322) extend across at least a portion of distal face (316). Alternatively, distal face (316) may incorporate electrodes (320, 322) in any other suitable fashion. In some scenarios, the cylindraceous shape of end effector (300) may make end effector (300) particularly suitable for use in the pulmonary vein such as, for example, isolating the pulmonary vein or performing a focused ablation for only a portion of the vein as well as other suitable anatomy of the organ.

### B. Example of End Effector with Frusto-Conical Shape

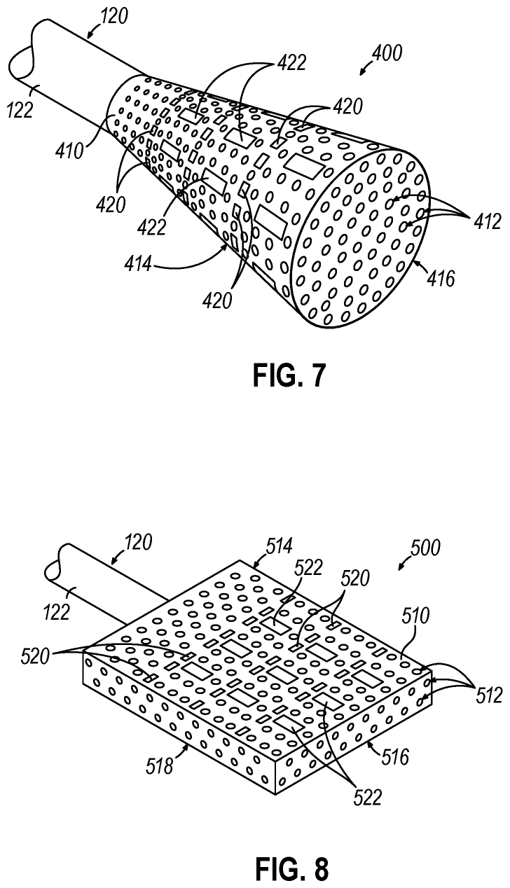

FIG. 7 shows another example of an end effector (400) located at distal end (124) of catheter (120) in place of end effector (200). End effector (400) of this example may be configured and operable just like end effector (200) described above, except for the differences described below. Like end effector (200), end effector (400) of this example includes an inflatable body (410) (e.g., in the form of an expandable membrane), a set of mapping electrodes (420), and a set of ablation electrodes (422). While not shown, end effector (400) may further include a central shaft like central shaft (126); and in some versions, may further include reference electrodes like reference electrodes (128, 230). Mapping electrodes (420) are arranged in generally circumferential arrays that extend along respective latitudinal paths, with such latitudinal paths being longitudinally spaced apart from each other; and are otherwise configured and operable just like mapping electrodes (220). Ablation electrodes (422) are arranged in generally longitudinal arrays that extend along respective longitudinal paths, with such longitudinal paths being angularly spaced apart from each other; and are otherwise configured and operable just like ablation electrodes (222).

By way of example only, electrodes (420, 422) may be formed of nitinol, platinum, gold, or any other suitable material. In some versions, electrodes (420, 422) are formed of an extensible material and are thereby configured to expand with body (410). Electrodes (420, 422) may be applied directly to body (410) using a physical vapor deposition (PVD) process, sputter deposition, chemical vapor deposition (CVD), thermal deposition, or any other suitable process.

Like inflatable body (210), inflatable body (410) includes openings (412) and is operable to transition between a non-expanded state and an expanded state. However, unlike inflatable body (210), inflatable body (410) has a frusto-conical shape when in the expanded state. In this example, electrodes (420, 422) are only on the tapered portion (414) of inflatable body (410), such that electrodes (420, 422) do not extend along a flat distal face (416) of inflatable body (410). In some other versions, electrodes (420, 422) extend across at least a portion of distal face (416). Alternatively, distal face (416) may incorporate electrodes (420, 422) in any other suitable fashion. In some scenarios, the frusto-conical shape of end effector (400) may make end effector (400) particularly suitable for adaptation to varying geometries and diameters of pulmonary veins.

### C. Example of End Effector with Generally Flat Rectangular Shape

FIG. 8 shows another example of an end effector (500) located at distal end (124) of catheter (120) in place of end effector (200). End effector (500) of this example may be configured and operable just like end effector (200) described above, except for the differences described below. Like end effector (200), end effector (500) of this example includes an inflatable body (510) (e.g., in the form of an expandable membrane), a set of mapping electrodes (520), and a set of ablation electrodes (522). While not shown, end effector (500) may further include a central shaft like central shaft (126); and in some versions, may further include reference electrodes like reference electrodes (128, 230). Mapping electrodes (520) are arranged in generally lateral arrays that extend along respective laterally oriented paths, with such laterally oriented paths being longitudinally spaced apart from each other; and are otherwise configured and operable just like mapping electrodes (220). Ablation electrodes (522) are arranged in generally longitudinal arrays that extend along respective longitudinal paths, with such longitudinal paths being laterally spaced apart from each other; and are otherwise configured and operable just like ablation electrodes (222).

By way of example only, electrodes (520, 522) may be formed of nitinol, platinum, gold, or any other suitable material. In some versions, electrodes (520, 522) are formed of an extensible material and are thereby configured to expand with body (510). Electrodes (520, 522) may be applied directly to body (510) using a physical vapor deposition (PVD) process, sputter deposition, chemical vapor deposition (CVD), thermal deposition, or any other suitable process.

Like inflatable body (510), inflatable body (510) includes openings (512) and is operable to transition between a non-expanded state and an expanded state. However, unlike inflatable body (210), inflatable body (510) has a generally flat rectangular shape when in the expanded state. In this example, electrodes (520, 522) are only on the broadest face (514) of inflatable body (510), such that electrodes (520, 522) do not extend along a flat distal face (516) or lateral sides (518) of inflatable body (510). In some other versions, electrodes (520, 522) extend across at least a portion of distal face (516) or lateral sides (518). Alternatively, distal face (516) or lateral sides (518) may incorporate electrodes (520, 522) in any other suitable fashion. In some scenarios, the generally flat rectangular shape of end effector (500) may make end effector (500) particularly suitable for recording signals along chamber walls, with the geometry allowing for inclusion of a grid-like electrode pattern on the broadest face. The grid-like pattern of electrodes may enable bipolar signal recordings in two orthogonal directions.

### IV. Example of End Effector Having Metallic Lattice Structure

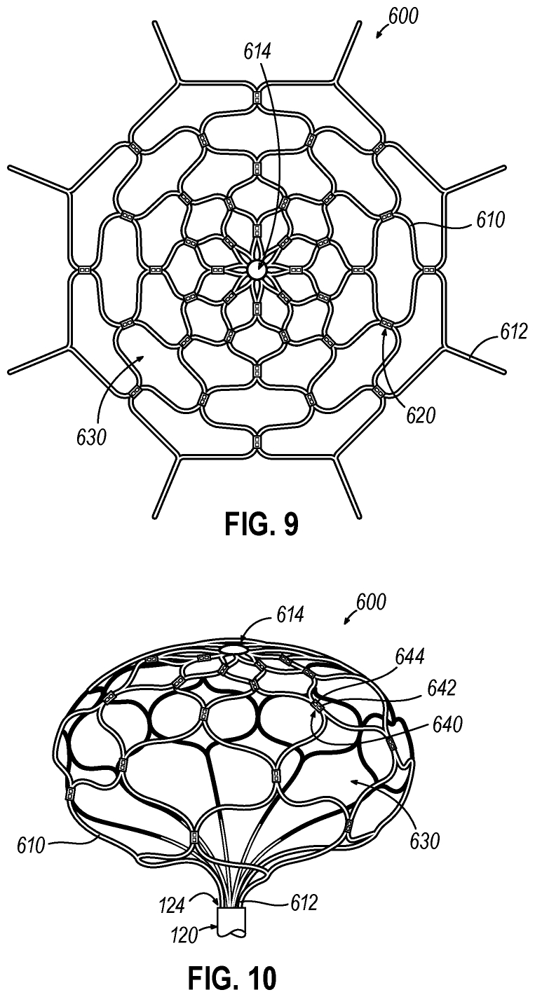

FIGS. 9-10 shows another example of a lattice structure (600) that may be combined with a membrane (e.g., like inflatable body (210)) to form an end effector; or may form an end effector by itself. Lattice structure (600) of this example is configured to be initially constructed in a flat configuration as shown in FIG. 9; then folded into a bulbous or generally spherical shape as shown in FIG. 10. In versions where lattice structure (600) is combined with a membrane, the membrane may be positioned on the interior of the generally spherical shape that is formed by lattice structure (600) when lattice structure (600) is in the folded configuration shown in FIG. 10. Alternatively, the membrane may be provided in discrete segments that are positioned in each opening (630) defined by lattice structure (600). In either case, the membrane may function like an inflatable body, such that the membrane may assist in driving lattice structure from a non-expanded state (e.g., similar to the state of end effector (200) shown in FIG. 2A) to the expanded state (e.g., to achieve the generally spherical configuration shown in FIG. 10) through inflation. In versions where a membrane is included, the membrane may further include openings like openings (212) described above; or may omit such openings.

Some versions of lattice structure (600) may omit a membrane altogether. In such versions, as well as in some versions where a membrane is included, lattice structure (600) may be resiliently biased to assume the generally spherical configuration shown in FIG. 10. For instance, lattice structure (600) may include nitinol or some other resilient material to bias lattice structure (600) to the generally spherical configuration shown in FIG. 10. In either case, lattice structure (600) may nevertheless be compressible to achieve a non-expanded configuration similar to what is shown in end effector (200) of FIG. 2A. By way of example only, lattice structure (600) may be compressible to achieve a size less than or equal to approximately 6 French when in the non-expanded configuration.

Lattice structure (600) is formed by a plurality of curved strip bodies (610). Each strip body (610) has an undulating curved configuration. The proximal end (612) of each strip body (610) contacts the proximal end (612) of an adjacent strip body (610) as best seen in FIG. 9 to form a pair of proximal ends (612). Proximal ends (612) secure lattice structure (600) to catheter (120) as shown in FIG. 10. Adjacent strip bodies (610) also contact each other at node regions (620). In some versions, adjacent strip bodies (610) overlap each other at node regions (620). Openings (630) are defined between adjacent strip bodies (610). The distal ends of strip bodies (610) converge at a distal end (614) of lattice structure (600). When lattice structure (600) is in the flat configuration as shown in FIG. 9, this distal end (614) is in the center of the flat configuration. By way of example only, distal end (614) or other portions of lattice structure (600) may be configured and operable in accordance with at least some of the teachings of U.S. Pub. No. 2015/0342532, entitled “High Electrode Density Basket Catheter,” published Dec. 3, 2015, the disclosure of which is incorporated by reference herein, in its entirety; U.S. Pub. No. 2017/0071543, entitled “Convertible Basket Catheter,” published Mar. 16, 2017, the disclosure of which is incorporated by reference herein, in its entirety; or U.S. Pub. No. 2017/0347959, entitled “Spine Construction for Basket Catheter,” published Dec. 7, 2017, the disclosure of which is incorporated by reference herein, in its entirety.

As shown in FIG. 10, each node region (620) includes an electrode pair (640). Each electrode pair (640) includes a first electrode (642) and a second electrode (644). Electrodes (642, 644) may be printed on strip body (610) or otherwise be integrated into strip body (610). Electrodes (642, 644) are configured to provide EP mapping by contacting tissue and picking up potentials from the contacted tissue (e.g., to provide an electrocardiogram signal). In other words, each electrode pair (640) is configured to provide bipolar sensing of electrocardiogram signals as electrode pair (640) is placed in contact with cardiovascular tissue. Thus, each electrode pair (640) may be considered as collectively forming a single “sensor.” Each electrode (642, 644) may be coupled with a corresponding trace or other electrical conduit of lattice structure (600), thereby enabling signals picked up by electrode pairs (640) to be communicated back through electrical conduits (not shown) in catheter (120) to console (12), which may process the signals to provide EP mapping to thereby identify locations of aberrant electrical activity within the cardiac anatomy. This may in turn allow the physician (PH) to identify the most appropriate regions of cardiac tissue to ablate (e.g., with electrical energy, cryoablation, etc.), to thereby prevent or at least reduce the communication of aberrant electrical activity across the cardiac tissue.

FIG. 11 shows another example of an arrangement where reference electrodes (646) are positioned in an opposing fashion in relation to a mapping electrode (642). While FIG. 11 only shows mapping electrode (642), a similar arrangement may be provided with respect to mapping electrode (644). As shown, mapping electrode (642) is applied over a dielectric layer (650). Dielectric layer (650) is applied over a biocompatible structural layer (652). By way of example only, biocompatible structural layer (652) may include platinum or any other suitable biocompatible metal. Biocompatible structural layer (652) is applied over a dielectric insulating layer (654). A conductive layer trace (656) is positioned under dielectric insulating layer (654). A via (660) provides a path for signals from mapping electrode (642) to be communicated to conductive layer trace (656). Conductive layer trace (656) may form part of the path by which potentials picked up by mapping electrode (642) are communicated back to console (12). Mapping electrode (644) may have its own dedicated region of conductive trace layer (656) that is insulated from the region of conductive trace layer (656) that is dedicated to mapping electrode (644), such that mapping electrodes (642, 644) have their own respective discrete regions of conductive trace layer (656). Another dielectric layer (658) is positioned under conductive trace layer (656). Dielectric layer (658) is positioned over strip body (610). As noted above, strip body (610) may be in the form of a nitinol thin film.

As also shown in FIG. 11, the underside of strip body (610) (which would be facing the interior region of end effector (600)) includes a dielectric layer (674), a conductive trace layer (672), a dielectric insulating layer (670), and a reference electrode (646). Reference electrode (646) may operate similar to reference electrodes (128, 230) described above, such that reference electrode (646) may be utilized to pick up reference potentials from blood or saline that passes through the interior of end effector (600) via openings (630) during an EP mapping procedure. Since reference electrodes (646) would be positioned within the interior of end effector (600), strip bodies (610) will prevent tissue from contacting reference electrodes (646) during use of end effector (600) in an EP mapping procedure; while still allowing blood and saline to flow freely through end effector (600) to reach reference electrodes (646). A via (676) provides a path for signals from reference electrode (646) to be communicated to conductive trace layer (672). Conductive trace layer (672) forms part of the path for signals picked up by reference electrode (646) to reach console (12). In some versions, just one single reference electrode (646) is positioned opposite to each electrode pair (640). Alternatively, reference electrodes (646) may have any other suitable spatial or structural relationships with electrodes (642, 644).

In versions where end effector (600) includes ablation capabilities, biocompatible structural layer (652) may effectively form ablation electrodes. By providing a substantial portion of the exposed surface area of end effector (600), layer (652) may generate larger lesions than would otherwise be generated using small individual electrodes.

In the example shown in FIG. 11, all the layers (642, 650, 652, 654, 656, 658, 660) shown on the exterior side of strip body (610) may be applied to strip body (610) using a physical vapor deposition (PVD) process, sputter deposition, chemical vapor deposition (CVD), thermal deposition, or any other suitable process. Similarly, all the layers (646, 670, 672, 674, 676) shown on the interior side of strip body (610) may be applied to strip body (610) using a physical vapor deposition (PVD) process, sputter deposition, chemical vapor deposition (CVD), thermal deposition, or any other suitable process.

In some variations of end effector (600), an insulating layer may be provided on the entire exposed surface of each strip body (610), with cutouts formed in the insulating layer to expose electrodes (642, 644, 646). Such an insulating layer may effectively form recesses at the cutouts in which electrodes (642, 644, 646) are disposed. By forming such recesses for electrodes (642, 644, 646), the insulating layer may mechanically protect electrodes (642, 644, 646). Moreover, in some such versions, it may be unnecessary to form any vias that couple electrodes (642, 644, 646) with corresponding traces. In other words, each electrode (642, 644, 646) and its corresponding trace may be on the same layer.

### V. Examples of Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An apparatus comprising: (a) a catheter, at least a portion of the catheter being sized and configured to fit within a lumen of a cardiovascular system; and (b) an end effector positioned at a distal end of the catheter, the end effector comprising: (i) an expandable body, the expandable body being configured to transition between a non-expanded state and an expanded state, the expandable body having an inner surface and an outer surface, the expandable body defining a plurality of openings extending from the inner surface to the outer surface, and (ii) a plurality of electrodes deposited on the outer surface of the expandable body, the electrodes being configured to expand with the expandable body from the non-expanded state to the expanded state, the electrodes including one or more electrodes selected from the group consisting of: (A) mapping electrodes that are configured to sense electrical potentials in tissue contacting the mapping electrodes, and (B) ablation electrodes that are operable to ablate tissue contacting the ablation electrodes.

### Example 2

The apparatus of Example 1, the expandable body comprising a membrane.

### Example 3

The apparatus of Example 2, the membrane being extensible.

### Example 4

The apparatus of any one or more of Examples 1 through 3, the openings being configured to expel irrigation fluid from an interior region defined by the expandable body.

### Example 5

The apparatus of any one or more of Examples 1 through 4, the openings being configured to allow passage of fluid into an interior region defined by the expandable body.

### Example 6

The apparatus of any one or more of Examples 1 through 5, the expandable body being configured to define a bulbous shape in the expanded state.

### Example 7

The apparatus of Example 6, the bulbous shape being generally spherical.

### Example 8

The apparatus of any one or more of Examples 1 through 5, the expandable body being configured to define a cylindraceous shape in the expanded state.

### Example 9

The apparatus of any one or more of Examples 1 through 5, the expandable body being configured to define a frusto-conical shape in the expanded state.

### Example 10

The apparatus of any one or more of Examples 1 through 5, the expandable body being configured to define a rectangular shape in the expanded state.

### Example 11

The apparatus of any one or more of Examples 1 through 10, the end effector further comprising one or more resilient members, the one or more resilient members being configured to urge the expandable body toward the expanded state.

### Example 12

The apparatus of Example 11, the one or more resilient members comprising one or more resilient strips.

### Example 13

The apparatus of any one or more of Examples 11 through 12, the one or more resilient members comprising nitinol.

### Example 14

The apparatus of any one or more of Examples 11 through 13, the one or more resilient members being deposited on the inner surface or the outer surface of the expandable body.

### Example 15

The apparatus of any one or more of Examples 1 through 14, the plurality of electrodes comprising a plurality of mapping electrodes and a plurality of ablation electrodes.

### Example 16

The apparatus of any one or more of Examples 1 through 15, the end effector further comprising at least one reference electrode.

### Example 17

The apparatus of Example 16, the at least one reference electrode being disposed on the inner surface of the expandable body.

### Example 18

The apparatus of Example 16, the end effector further comprising a central shaft, the at least one reference electrode being disposed on the central shaft.

### Example 19

The apparatus of any one or more of Examples 1 through 18, the expandable body comprising a resilient lattice structure.

### Example 20

The apparatus of Example 19, the resilient lattice structure being formed by a plurality of curved resilient strips.

### Example 21

The apparatus of Example 20, the curved resilient strips including regions overlapping with each other.

### Example 22

The apparatus of Example 21, at least some of the plurality of electrodes being located at the regions of the resilient strips overlapping with each other.

### Example 23

The apparatus of any one or more of Examples 19 through 22, the resilient lattice structure comprising nitinol.

### Example 24

The apparatus of any one or more of Examples 1 through 23, further comprising a processor in communication with the electrodes.

### Example 25

The apparatus of Example 24, the electrodes including mapping electrodes that are configured to sense electrical potentials in tissue contacting the mapping electrodes, the processor being operable to process potentials picked up by the mapping electrodes.

### Example 26

The apparatus of Example 25, the processor being operable to provide an electrocardiogram reading based on potentials picked up by the mapping electrodes.

### Example 27

The apparatus of any one or more of Examples 24 through 26, the electrodes including ablation electrodes that are operable to ablate tissue contacting the ablation electrodes, processor being operable to drive activation of the ablation electrodes with electrical energy.

### Example 28

The apparatus of any one or more of Examples 1 through 27, further comprising a position sensor, the position sensor being operable to generate signals indicating a position of the end effector in three-dimensional space.

### Example 29

The apparatus of Example 28, the position sensor being located on the end effector.

### Example 30

The apparatus of Example 29, the position sensor being located on the expandable body.

### Example 31

An apparatus comprising: (a) a catheter, at least a portion of the catheter being sized and configured to fit within a lumen of a cardiovascular system; and (b) an end effector positioned at a distal end of the catheter, the end effector comprising: (i) an expandable membrane, the expandable membrane being configured to transition between a non-expanded state and an expanded state, the expandable membrane having an inner surface and an outer surface, the expandable membrane defining a plurality of openings extending from the inner surface to the outer surface, and (ii) a plurality of electrodes deposited on the outer surface of the expandable membrane, the electrodes being configured to expand with the expandable membrane from the non-expanded state to the expanded state, the electrodes including one or more electrodes selected from the group consisting of: (A) mapping electrodes that are configured to sense electrical potentials in tissue contacting the mapping electrodes, and (B) ablation electrodes that are operable to ablate tissue contacting the ablation electrodes.

### Example 32

An apparatus comprising: (a) a catheter, at least a portion of the catheter being sized and configured to fit within a lumen of a cardiovascular system; and (b) an end effector positioned at a distal end of the catheter, the end effector comprising: (i) an expandable lattice structure, the expandable lattice structure being configured to transition between a non-expanded state and an expanded state, the expandable lattice structure comprising a plurality of strips and defining a plurality of openings between the strips, and (ii) a plurality of electrodes deposited on the expandable lattice structure, the electrodes including one or more electrodes selected from the group consisting of: (A) mapping electrodes that are configured to sense electrical potentials in tissue contacting the mapping electrodes, and (B) ablation electrodes that are operable to ablate tissue contacting the ablation electrodes.

### Example 33

A method comprising: (a) providing an expandable body, the expandable body being configured to transition between an expanded state and a non-expanded state, the expandable body being configured to fit within a lumen of a cardiovascular system in the non-expanded state; (b) depositing a plurality of electrodes onto a surface of the expandable body, the electrodes and expandable body together defining an end effector, the electrodes being configured to expand with the expandable body from the non-expanded state to the expanded state, the electrodes including one or more electrodes selected from the group consisting of: (i) mapping electrodes that are configured to sense electrical potentials in tissue contacting the mapping electrodes, and (ii) ablation electrodes that are operable to ablate tissue contacting the ablation electrodes; and (c) securing the end effector to a distal end of a catheter shaft assembly.

### Example 34

The method of Example 33, the expandable body being initially formed as a planar structure, the planar structure being folded into a non-planar shape to further define the end effector.

### Example 35

The method of Example 34, the electrodes being deposited on the planar structure before the planar structure is folded into the non-planar shape.

### Example 36

The method of Example 33, the expandable body comprising a membrane, the electrodes being deposited directly on the membrane.

### Example 37

The method of any one or more of Examples 33 through 36, the depositing a plurality of electrodes onto a surface of the expandable body comprising utilizing a vapor deposition process.

### Example 38

The method of Example 37, the vapor deposition process comprising a physical vapor deposition process.

### Example 39

The method of any one or more of Examples 37 through 38, the vapor deposition process comprising a chemical vapor deposition process.

### Example 40

The method of any one or more of Examples 33 through 39, the depositing a plurality of electrodes onto a surface of the expandable body comprising utilizing a sputter deposition process.

### Example 41

The method of any one or more of Examples 33 through 40, the depositing a plurality of electrodes onto a surface of the expandable body comprising utilizing a thermal deposition process.

### Example 42

The method of any one or more of Examples 33 through 41, the deposited electrodes being formed of a resilient material.

### Example 43

The method of any one or more of Examples 33 through 42, the deposited electrodes being formed of an extensible material.

### Example 44

The method of any one or more of Examples 33 through 43, the deposited electrodes being formed of nitinol.

### Example 45

The method of any one or more of Examples 33 through 44, the expandable body comprising a first surface and a second surface, the second surface being opposite to the first surface, the depositing a plurality of electrodes onto a surface of the expandable body comprising: (i) depositing at least one electrode on the first surface of the expandable body, and (ii) depositing at least one electrode on the second surface of the expandable body.

### Example 46

The method of Example 45, the end effector including an interior region and an exterior region, the first surface being on the interior region of the end effector, the second surface being on the exterior region of the end effector.

### Example 47

An apparatus comprising: (a) a catheter, at least a portion of the catheter being sized and configured to fit within a lumen of a cardiovascular system; and (b) an end effector positioned at a distal end of the catheter, the end effector comprising: (i) an expandable membrane, the expandable membrane being configured to transition between a non-expanded state and an expanded state, the expandable membrane having an inner surface and an outer surface, the expandable membrane defining a plurality of openings extending from the inner surface to the outer surface, the openings being configured to allow fluid to pass through the membrane, and (ii) a plurality of electrodes deposited on the outer surface of the expandable membrane, the electrodes being configured to expand with the expandable membrane from the non-expanded state to the expanded state, the electrodes including one or more electrodes selected from the group consisting of: (A) mapping electrodes that are configured to sense electrical potentials in tissue contacting the mapping electrodes, and (B) ablation electrodes that are operable to ablate tissue contacting the ablation electrodes.

### VI. Miscellaneous

Any of the instruments described herein may be cleaned and sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, hydrogen peroxide, peracetic acid, and vapor phase sterilization, either with or without a gas plasma, or steam.

It should be understood that any of the examples described herein may include various other features in addition to or in lieu of those described above. By way of example only, any of the examples described herein may also include one or more of the various features disclosed in any of the various references that are incorporated by reference herein in its entirety.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

It should be appreciated that any patent, publication, or other disclosure material, in whole or in part, that is said to be incorporated by reference herein in its entirety is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein in its entirety, but which conflicts with existing definitions, statements, or other disclosure material set forth herein will only be incorporated to the extent that no conflict arises between that incorporated material and the existing disclosure material.

Having shown and described various versions of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. An apparatus comprising:
(a) a catheter, at least a portion of the catheter being sized and configured to fit within a lumen of a cardiovascular system; and
(b) an end effector positioned at a distal end of the catheter, the end effector comprising:
(i) an expandable body, the expandable body being configured to transition between a non-expanded state and an expanded state, the expandable body having an inner surface and an outer surface, the expandable body defining a plurality of openings extending from the inner surface to the outer surface, and
(ii) a plurality of electrodes deposited on the outer surface of the expandable body, the electrodes being configured to expand with the expandable body from the non-expanded state to the expanded state, the electrodes including one or more electrodes selected from the group consisting of:
(A) mapping electrodes that are configured to sense electrical potentials in tissue contacting the mapping electrodes, and
(B) ablation electrodes that are operable to ablate tissue contacting the ablation electrodes.

2. The apparatus of claim 1, the expandable body comprising a membrane.

3. The apparatus of claim 1, the openings being configured to expel irrigation fluid from an interior region defined by the expandable body.

4. The apparatus of claim 1, the openings being configured to allow passage of fluid into an interior region defined by the expandable body.

5. The apparatus of claim 1, the expandable body being configured to define a bulbous shape in the expanded state.

6. The apparatus of claim 1, the expandable body being configured to define a cylindraceous shape in the expanded state.

7. The apparatus of claim 1, the expandable body being configured to define a frusto-conical shape in the expanded state.

8. The apparatus of claim 1, the expandable body being configured to define a rectangular shape in the expanded state.

9. The apparatus of claim 1, the end effector further comprising one or more resilient members, the one or more resilient members being configured to urge the expandable body toward the expanded state.

10. The apparatus of claim 9, the one or more resilient members comprising one or more resilient strips.

11. The apparatus of claim 9, the one or more resilient members being deposited on the inner surface or the outer surface of the expandable body.

12. The apparatus of claim 1, the plurality of electrodes comprising a plurality of mapping electrodes and a plurality of ablation electrodes.

13. The apparatus of claim 1, the end effector further comprising at least one reference electrode.

14. The apparatus of claim 13, the at least one reference electrode being disposed on the inner surface of the expandable body.

15. The apparatus of claim 1, the expandable body comprising a resilient lattice structure.

16. The apparatus of claim 15, the resilient lattice structure being formed by a plurality of curved resilient strips.

17. The apparatus of claim 16, the curved resilient strips including regions overlapping with each other.

18. The apparatus of claim 17, at least some of the plurality of electrodes being located at the regions of the resilient strips overlapping with each other.

19. An apparatus comprising:
(a) a catheter, at least a portion of the catheter being sized and configured to fit within a lumen of a cardiovascular system; and
(b) an end effector positioned at a distal end of the catheter, the end effector comprising:
(i) an expandable membrane, the expandable membrane being configured to transition between a non-expanded state and an expanded state, the expandable membrane having an inner surface and an outer surface, the expandable membrane defining a plurality of openings extending from the inner surface to the outer surface, the openings being configured to allow fluid to pass through the membrane, and
(ii) a plurality of electrodes deposited on the outer surface of the expandable membrane, the electrodes being configured to expand with the expandable membrane from the non-expanded state to the expanded state, the electrodes including one or more electrodes selected from the group consisting of:
(A) mapping electrodes that are configured to sense electrical potentials in tissue contacting the mapping electrodes, and
(B) ablation electrodes that are operable to ablate tissue contacting the ablation electrodes.

20. An apparatus comprising:
(a) a catheter, at least a portion of the catheter being sized and configured to fit within a lumen of a cardiovascular system; and
(b) an end effector positioned at a distal end of the catheter, the end effector comprising:
(i) an expandable lattice structure, the expandable lattice structure being configured to transition between a non-expanded state and an expanded state, the expandable lattice structure comprising a plurality of strips and defining a plurality of openings between the strips, and
(ii) a plurality of electrodes deposited on the expandable lattice structure, the electrodes including one or more electrodes selected from the group consisting of:
(A) mapping electrodes that are configured to sense electrical potentials in tissue contacting the mapping electrodes, and
(B) ablation electrodes that are operable to ablate tissue contacting the ablation electrodes.
